(19) United States
(12) Patent Application Publication
(10) Pub. No.: US 2015/0323539 A1
(43) Pub. Date: Nov. 12, 2015

(54) BLOOD MARKERS FOR LUNG CANCER PREDISPOSITION

(71) Applicants: THE REGENTS OF THE UNIVERSITY OF CALIFORNIA, Oakland, CA (US); THE UNITED STATES GOVERNMENT REPRESENTED BY THE DEPARTMENT OF VETERANS AFFAIRS, Washington, DC (US)

(72) Inventors: Robert H. Schiestl, Encino, CA (US); Aaron M. Chapman, LaPlace, LA (US); Steven M. Dubinett, Los Angeles, CA (US)

(73) Assignees: THE REGENTS OF THE UNIVERSITY OF CALIFORNIA, OAKLAND, CA (US); THE UNITED STATES GOVERNMENT REPRESENTED BY THE DEPARTMENT OF VETERANS AFFAIRS, Washington, DC (US)

(21) Appl. No.: 14/708,213

(22) Filed: May 9, 2015

Related U.S. Application Data
(60) Provisional application No. 61/990,866, filed on May 9, 2014.

(51) Int. Cl. G01N 33/574

(52) U.S. Cl. CPC G01N 33/57423; G01N 2800/7009; G01N 2800/7095

(57) ABSTRACT

The invention provides a method for detection of lung cancer, or predisposition to lung cancer, in a subject that comprises assaying a test sample of peripheral blood from the subject for a marker of DNA damage. An elevated amount of marker present in the test sample compared to control sample is indicative of lung cancer, or predisposition to lung cancer. The method can be adapted for quantitatively monitoring the efficacy of treatment of lung cancer in a subject. Markers of DNA damage include single- and/or double-stranded breaks in leukocytes, oxidative DNA damage in leukocytes, or a marker of nitrotyrosine oxidative activity (protein nitrosylation in leukocytes). This unexpected discovery of markers of systemic genotoxicity that can be tested using circulating leukocytes enables detection of lung cancer, or predisposition to lung cancer, with a relatively simple and minimally invasive assay using peripheral blood.

This application claims the benefit of U.S. provisional application No. 61/990,866, filed May 9, 2014, the entire contents of which are incorporated herein by reference.

### STATEMENT REGARDING FEDERALLY SPONSORED RESEARCH

This invention was made with Government support under A1094756, CA152751, awarded by the National Institutes of Health. The Government has certain rights in the invention.

This work was supported by the U.S. Department of Veterans Affairs, and the Federal Government has certain rights in the invention.

### TECHNICAL FIELD OF THE INVENTION

The present invention relates generally to detection, diagnosis, and monitoring of lung cancer, including the detection of a predisposition to lung cancer. The invention more specifically pertains to use of systemic genotoxicity as a marker for lung cancer predisposition.

### BACKGROUND OF THE INVENTION

Lung cancer is the leading cause of death from cancer in both men and women in the United States and causes more deaths than the next three most common cancers combined (colon, breast, and prostate). Nearly 90 percent of individuals who develop lung cancer are smokers, yet only 10-15 percent of lifetime smokers will develop the disease. This statistic suggests that some individuals are more susceptible to developing lung cancer than others. What is further perplexing is the notion that it is not well understood why some individuals are more susceptible to smoking induced lung cancer. This suggests that other factors concomitant with history of smoking may be involved in susceptibility to develop carcinogenesis in these select individuals. It is well documented that factors such as inflammation, other confounding diseases, age, genetic polymorphisms, race, sex, family history of cancer, and other environmental exposures may play a major contributing role factor to cancer incidence in these individuals. Furthermore, there are few phenotypic tools available to determine susceptibility to lung cancer opening the door to pioneering new technologies in disease susceptibility and early prognosis

There is a need to identify improved markers for lung cancer predisposition. There is also a need for methods of detecting lung cancer and monitoring treatment efficacy.

### SUMMARY OF THE INVENTION

The invention is based on the discovery that markers of DNA double strand breaks, oxidative DNA damage, and inflammation induced protein damage provide phenotypic tools to assess if individuals are more susceptible to cigarette smoke extract induced genotoxicity. Described herein are statistically significant results correlating age and race to susceptibility to develop DNA damage and lung cancer difference to non-lung cancer, as well as positive yet non-significant trends in gender, family history of cancer, pack years of cigarettes smoked, and individuals with history of other cancers.

The data presented herein thus establish that genotoxicity present in peripheral leukocytes can be utilized as a biomarker to predict lung cancer susceptibility. Using bi- and multi-variate models, we were able to assess various interactions to determine the contribution to cigarette smoke extract induced genotoxicity. Even with a small sample size, the results of our study show significant interactions of age and race on cigarette smoke extract induced DNA damage as well as the difference between cancer and non-cancer individuals, and positive trends in sex, previous personal cancer history, family history of cancer, and smoking history that is associated with lung cancer. One interesting observation that was found was, in two of our biomarkers, individuals who did not have a smoking history were highly susceptible to cigarette smoke extract induced DNA damage. This Information could lead to the pursuit of elucidating what could cause non-smoking Individuals to be susceptible to cigarette smoke extract induced DNA damage. A significant difference was observed between cancer and non-cancer individuals, in particular such patients that were in their 40 and 50s. This finding could be applied for a blood test for lung cancer predisposition regardless of smoking history, as the non-smoking lung cancer patient showed the highest sensitivity.

The invention provides a method for detection of lung cancer, or predisposition to lung cancer, in a subject. In one embodiment, the method comprises assaying a test sample of peripheral blood from the subject for a marker of DNA damage. The assaying can comprise incubating a test sample of peripheral leukocytes from the subject with cigarette smoke extract; and assaying the test sample for a marker of DNA damage. The amount of marker present in the test sample is then compared to that present in a control sample. The method further comprises determining the presence of cancer or predisposition to lung cancer when an increased amount of the marker is present in the test sample compared to the control sample. The method can be adapted for quantitatively monitoring the efficacy of treatment of lung cancer in a subject. An elevated amount of marker present in the test sample compared to the control sample is indicative of lung cancer, or failure to respond to treatment. In some embodiments, the method further comprises prescribing treatment for lung cancer or modifying an ongoing treatment strategy on the basis of the assay results.

In one embodiment, the marker of DNA damage is single- and/or double-stranded breaks in leukocytes. Such strand breaks can be detected by immunoassay for γ-H2AX and/or an alkaline comet assay. In another embodiment, the marker of DNA damage is oxidative DNA damage in leukocytes, or a marker of nitric oxide oxidative activity (protein nitrosylation in leukocytes). Oxidative DNA damage can be assayed via an enzyme hOgg1-modified comet assay or by immunoassay for 8-oxoguanine. An underlying oxidative process (nitric oxide-mediated oxidation) can be assayed by immunoassay for protein nitrotyrosine. In a further embodiment, the marker of DNA damage is micronuclei formation in mature, normochromatic erythrocytes. In one embodiment, the assaying or measuring comprises an immunoassay for γ-H2AX, nitrotyrosine, or 8-oxoguanine.

The invention additionally provides kits for use in carrying out the methods described herein. In one embodiment, the kit comprises reagents for detecting γ-H2AX, nitrotyrosine, and/or 8-oxoguanine. In one embodiment, the reagents are antibodies. In one embodiment, the kit further comprises cigarette smoke extract.

### BRIEF DESCRIPTION OF THE FIGURES

FIG. 1. The assessment of the bi-variant interaction between time of cigarette smoke extract incubation and the age of the individual. This depicts the role age has on susceptibility to develop γH2AX foci in peripheral leukocytes. Individuals who are in their 40's, N=2 (uppermost line), N=4 (50's, 2nd line), N=8 (60's, middle line), N=6 (70's, 2^{nd} line from bottom), and N=3 (80's, lowermost line), respectively. p<0.0247

FIG. 2. The assessment of the bi-variant interaction between time of cigarette smoke extract incubation and age of the individual. This depicts the role age has on susceptibility to develop 8-oxoguanine positive cells in peripheral leukocytes. Individuals who are in their 40's, N=2 (uppermost line), N=4 (50's, 2nd line), N=8 (60's, middle line), N=6 (70's, 2^{nd} line from bottom), and N=3 (80's, lowermost line), respectively. P=0.0759

FIG. 3. The assessment of the bi-variant interaction between time of cigarette smoke extract incubation and smoking history of the individual. This depicts the role race has on susceptibility to develop Nitrotryosine positive cells in peripheral leukocytes. White (lighter middle line) n=17, Black (lowermost line) n=3 Asian (lower, darker middle line) n=2, and Hispanic (uppermost line) n=1. p<0.0243

FIG. 4. Multi-variant modeling assessing models of interaction between time of cigarette smoke extract incubation and age while controlling for sex, race, and pack years smoked. n=2 individuals who were in their 40's (uppermost line), individuals who were in their 50's n=4 (2nd line), individuals who were in their 60's n=8 (middle line), individuals who were in their 70's n=6 (2^{nd} line from bottom), and individuals who were in their 80's n=3 (lowermost line). Significant positive interaction at p<0.0240 occurs between incubation time and age. A near statistically significant results in our controlled variable, race at p=0.0795.

FIG. 5. Multi-variate modeling assessing models of interaction between time of cigarette smoke extract incubation and age while controlling for sex, race, and pack years smoked. n=2 individuals who were in their 40's (dark blue line), individuals who were in their 50's n=4 (red line), individuals who were in their 60's n=8 (dark green line), individuals who were in their 70's n=6 (gold line), and individuals who were in their 80's n=3 (light blue line). Nearly significant positive interaction at p=0.0749 occurs between incubation time and age. A statistically significant result was found in our controlled variable, race at p<0.0012.

FIG. 6. Multi-variant modeling assessing models of interaction between time of cigarette smoke extract incubation and age while controlling for sex, race, and pack years smoked. n=2 individuals who were in their 40's (uppermost line), individuals who were in their 50's n=4 (2^{nd} line), individuals who were in their 60's n=8 (middle line), individuals who were in their 70's n=6 (2^{nd} line from bottom), and individuals who were in their 80's n=3 (lowermost line). p=0.2179 is between the time and age interaction. A statistically significant result was found in our controlled variable, race at p<0.0012.

FIG. 7. Multi-variant modeling assessing models of interaction between time of extract incubation and history of lung cancer while controlling for sex, race, and pack years smoked. Individuals with lung cancer (upper line at 0-6 hours; lower at 24 hours) n=17, individuals without lung cancer n=7 (lower line at 0-6 hours; upper at 24 hours). This interaction was trending towards significant at p=0.0971.

FIG. 8. Multi-variant modeling assessing models of interaction between time of extract incubation and history of lung cancer while controlling for sex, race, and pack years smoked. Individuals with lung cancer (upper line at 0-6 hours; lower line at 24 hours) n=17, individuals without lung cancer n=7 (lower line at 0-3 hours; upper line at 24 hours). This interaction was significant at p=0.0136.

FIG. 9. Multi-variant modeling assessing models of interaction between time of extract incubation with individuals who are 40-59 years of age and a history of lung cancer and individuals who are 40-59 years with no history of lung cancer while controlling for smoking history. Individuals with lung cancer (upper line) n=3, individuals with no lung cancer n=3 (lower line). This interaction was p=0.3282

FIG. 10. Multi-variant modeling assessing models of interaction between time of extract incubation with individuals who are 40-59 years of age and a history of lung cancer and individuals who are 40-59 years with no history of lung cancer while controlling for smoking history. Individuals with lung cancer (upper line at 0-3 hours; lower line at 6-24 hours) n=3, individuals with no lung cancer n=3 (lower line at 0-3 hours; upper line at 6-24 hours). This interaction was significant at p=0.0099.

FIG. 11. Multi-variant modeling assessing models of interaction between time of extract incubation with individuals who are 40-59 years of age and a history of lung cancer and individuals who are 40-59 years with no history of lung cancer while controlling for smoking history. Individuals with lung cancer (upper line) n=3, individuals with no lung cancer n=3 (lower line). This interaction is p=0.8293.

### DETAILED DESCRIPTION OF THE INVENTION

The invention described herein is based on the discovery that assays that detect systemic genotoxicity can be used to detect, diagnose and monitor lung cancer, or predisposition to lung cancer, and to guide in the prognosis and selection of treatment. Assays that detect a variety of endpoints for genotoxicity in response to cigarette smoke exposure in peripheral leukocytes have been found to correlate quantitatively with predisposition to lung cancer. These assays include immunostaining for γ-H2AX, which measures DNA double strand breaks, and the alkaline comet assay, which measures levels of DNA single and double strand breaks, as well as oxidative DNA base damage. DNA damage can also be measured by assaying micronucleus formation in normochromatic erythrocytes. This unexpected discovery of markers of genotoxicity present in circulating leukocytes, and their sensitivity to contact with cigarette smoke, enables detection of predisposition to lung cancer with a relatively simple and minimally invasive assay using peripheral blood.

### DEFINITIONS

All scientific and technical terms used in this application have meanings commonly used in the art unless otherwise specified. As used in this application, the following words or phrases have the meanings specified.

As used herein, a “sample” from a subject means a specimen obtained from the subject that contains blood or blood-derived cells. In a typical embodiment, the sample is peripheral blood or other sample containing peripheral leucocytes. For example, a sample of peripheral leukocytes can be obtained from peripheral blood.

As used herein, the term “subject” includes any human or non-human animal. The term “non-human animal” includes all vertebrates, e.g., mammals and non-mammals, such as non-human primates, horses, sheep, dogs, cows, pigs, chickens, amphibians, reptiles, etc.

As used herein, a “marker of DNA damage” includes single- and/or double-stranded DNA breaks, e.g., as detected by immunoassay for γ-H2AX and/or an alkaline comet assay, micronuclei formation, indicators of oxidative DNA damage, e.g., as detected via an enzyme hOgg1-modified comet assay, as well as indirect markers, such as nitrotyrosine, a marker of inflammation that has been shown to induce oxidative DNA damage. Immunoassays that detect γ-H2AX, nitrotyrosine, and/or 8-oxoguanine are examples of assays that detect a marker of DNA damage.

As used herein, a “control” can be a sample prepared and treated identically, but without exposure to cigarette smoke extract, or, where applicable, it can be an appropriate background reading, such as exemplified in the description below.

As used herein, “a” or “an” means at least one, unless clearly indicated otherwise.

### Methods of Detecting & Monitoring Lung Cancer or Predispostion to Lung Cancer

The invention provides a method for detection of lung cancer, or predisposition to lung cancer, in a subject. In one embodiment, the method comprises assaying a test sample of peripheral leukocytes from the subject for a marker of DNA damage after the leukocytes have been incubated with cigarette smoke extract. The incubation is typically for 3, 6 or 24 hours, but incubation periods of 1, 2, 3, 4, 5, 6, 8, 12, and 24 hours are contemplated. The amount of marker present in the test sample is then compared to that present in a control sample. An elevated amount of marker present in the test sample compared to the control sample is indicative of lung cancer, or predisposition to lung cancer.

The test sample is typically peripheral blood. Alternatively, the test sample can be bone marrow or body cavity fluids (such as peritoneal, pleural, synovial, or cerebrospinal fluids). DNA damage detected in peripheral blood leucocytes correlates with disease activity and with DNA damage in lymphoid organs, such as spleen, mesenteric lymph nodes and peripheral lymph nodes, and in intestinal epithelial cells. Test samples can be obtained from subjects using conventional means, such as venipuncture or capillary puncture. Normally the most desirable site for obtaining a blood sample for laboratory testing is from the veins of the antecubital fossa area, i.e. the bend of the elbow of the arm. A capillary puncture may be used when venipuncture would be too invasive or not possible. In general, capillary punctures may be done on earlobes, fingertips, heels, or toes, however, heels and toes are not a site of choice, especially in adults. Heel areas are typically used with neonates and younger infants. The site of choice in older children as well as adults is the distal lateral aspect of the fingertip; usually the second or third finger.

One can also assay DNA damage in subpopulations of leukocytes. In some embodiments, the leukocytes are lymphocytes, including subsets of lymphocytes, such as T cells, B cells, and/or NK cells. Also contemplated are monocytes, including subsets of monocytes, such as classical and pro-inflammatory monocytes. As one example, CD4+ and CD8+ T-cells, CD19+ B-cells, and CD11 b+ macrophages can be separated, such as by magnetic bead separation, for analysis. An increase in the diversity of cell types exhibiting DNA damage can be indicative of more severe or advanced disease.

In one embodiment, the marker of DNA damage is single- and/or double-stranded breaks in the cells to be analyzed. DNA strand breaks can be detected by immunoassay for γ-H2AX and/or an alkaline comet assay. One example of an immunoassay for γ-H2AX is an immunofluorescence assay using an antibody directed against γ-H2AX that is directly labeled, or that is used in conjunction with a labeled secondary antibody. Immunoreactive cells can be imaged using γH2AX, wherein cells having at least four distinct foci in the nucleus are considered positive. Apoptotic cells can be distinguished and excluded from the analysis. An example of an alkaline comet assay for measuring DNA damage in cells has been described by Olive et al. (Nat. Protocols 2006; 1(1):23-9). Comet images can be visualized, for example, using fluorescence microscopy, and analyzed using a CASP image analysis program. Tail length and fraction of DNA in the tail is represented in this assay by the olive tail moment.

In another embodiment, the marker of DNA damage is oxidative DNA damage in the cells to be analyzed. Oxidative DNA damage can be assayed via an enzyme hOgg1-modified comet assay. An example of an hOgg1 comet assay has been described by Smith et al. (Mutagenesis 2006; 21(3):185-90). In a further embodiment, the marker of DNA damage is micronuclei formation in mature, normochromatic erythrocytes, as described in the examples below and in Cancer Res. 2009; 69(11):4827-34; and Cancer Res. 2010; 70(5):1875-84.

In one example of bringing the leukocytes into contact with cigarette smoke extract, concentrated 40.3 puffs/ml cigarette smoke extract are diluted to a working solution of 5 parts/ml with PBS. 5 puffs/ml cigarette smoke extract (CSE) can be administered directly to whole peripheral blood to a final concentration of 1 puff/ml CSE and allowed to incubate in a shaking arc incubator, typically for 3, 6, or 24 hours. To determine the amount of DNA damage after CSE incubation, whole peripheral blood can be administered to erythrocyte lysis buffer, cells were laid over poly-D-lysine-coated coverslips and fixed with 4% paraformaldehyde (Electron Microscopy Sciences) at room temperature. Subsequently, cells are permeabilized and rinsed. After blocking, coverslips can be incubated with an antibody to H2AX, such as mouse anti-phospho-Histone H2A.X (JW301 Upstate) Temecula, Calif., and antibody to 8-oxoguanine, such as Mouse anti-8-oxoguanine clone 463.15 (Upstate, MAB3560), or an antibody to nitrotyrosine, such as Rabbit anti-nitrotyrosine (Upstate, 06-284). Known methods of detection can be employed, such as through the use of labeled secondary antibodies, to detect antibody binding.

Those skilled in the art will appreciate additional variations suitable for the method of detecting lung cancer, or predisposition to lung cancer, through detection of DNA damage in a specimen, as it provides remote monitoring (peripheral blood genotoxicity) to assess disease activity and response to treatment. This method can also be used to monitor levels of these markers in a sample from a patient undergoing treatment. The suitability of a therapeutic regimen for initial or continued treatment can be determined by monitoring marker levels using this method. The extent of genotoxicity present in a given patient or test sample can provide a prognostic indicator to guide treatment strategy. Accordingly, one can use information about the number and/or quantity of indicators present in a subject to assist in selecting an appropriate treatment protocol. If disease activity persists above an acceptable level, the clinician would consider increasing the treatment dose, or changing to a different therapeutic agent.

### Kits

For use in the diagnostic applications described herein, kits are also within the scope of the invention. Such kits can comprise a carrier, package or container that is compartmentalized to receive one or more containers such as vials, tubes, and the like, each of the container(s) comprising one of the separate elements to be used in the method. The antibodies of the kit may be provided in any suitable form, including frozen, lyophilized, or in a pharmaceutically acceptable buffer such as TBS or PBS. The kit may also include other reagents required for utilization of the reagents in vitro or in vivo such as buffers (i.e., TBS, PBS), blocking agents (solutions including nonfat dry milk, normal sera, Tween-20 Detergent, BSA, or casein), and/or detection reagents (i.e., goat anti-mouse IgG biotin, streptavidin-HRP conjugates, allophycocyanin, B-phycoerythrin, R-phycoerythrin, peroxidase, fluors (i.e., DyLight, Cy3, Cy5, FITC, HiLyte Fluor 555, HiLyte Fluor 647), and/or staining kits (i.e., ABC Staining Kit, Pierce)). The kits may also include other reagents and/or instructions for using antibodies and other reagents in commonly utilized assays described above such as, for example, flow cytometric analysis, ELISA, immunoblotting (i.e., western blot), in situ detection, immunocytochemistry, immunohistochemistry.

In one embodiment, the kit comprises reagents for detecting γ-H2AX, nitrotyrosine, and/or 8-oxoguanine. In one embodiment, the reagents are antibodies. In one embodiment, the kit further comprises cigarette smoke extract.

In one embodiment, the kit provides the reagent in purified form. In another embodiment, the reagents are immunoreagents that are provided in biotinylated form either alone or along with an avidin-conjugated detection reagent (i.e., antibody). In another embodiment, the kit includes a fluorescently labeled immunoreagent which may be used to directly detect antigen. Buffers and the like required for using any of these systems are well-known in the art and may be prepared by the end-user or provided as a component of the kit. The kit may also include a solid support containing positive- and negative-control protein and/or tissue samples. For example, kits for performing spotting or western blot-type assays may include control cell or tissue lysates for use in SDS-PAGE or nylon or other membranes containing pre-fixed control samples with additional space for experimental samples.

The kit of the invention will typically comprise the container described above and one or more other containers comprising materials desirable from a commercial and user standpoint, including buffers, diluents, filters, needles, syringes, and package inserts with instructions for use. In addition, a label can be provided on the container to indicate that the composition is used for a specific application, and can also indicate directions for use, such as those described above. Directions and or other information can also be included on an insert which is included with the kit.

### EXAMPLES

The following examples are presented to illustrate the present invention and to assist one of ordinary skill in making and using the same. The examples are not intended in any way to otherwise limit the scope of the invention.

### Example 1

### Blood Test for Lung Cancer Predisposition

This example demonstrates phenotypic tools to assess if individuals are more susceptible to cigarette smoke extract induced genotoxicity. To further strengthen our analysis, we combined our data with the known characteristics of confounding diseases, age, race, sex, family history of cancer, and other environmental exposures in each individual. In spite of our very small sample size we found statistically significant results correlating age and race to susceptibility to develop DNA damage and lung cancer difference to non-lung cancer, especially in the 40 and 50 year old patient group, as well as positive yet non-significant trends in gender, family history of cancer, pack years of cigarettes smoked, and individuals with history of other cancers. Thus, markers of genotoxicity detected in peripheral blood serve as markers for detection of predisposition to lung cancer.

Materials and Methods

Inclusion/Exclusion Criteria.

In the current study the amount of cigarette smoke extract induced genotoxicity was assessed in a heterogeneous cancer population of 30 patients comprising 24 former smokers 2 current smokers and 4 non-smokers. Inclusion criteria were as follows both men and women of all races and ethnic groups were eligible; individuals who were Age ≧18 years; had the ability to provide consent; had concurrent illness including COPD; had no known HIV or tuberculosis; non-smokers who have smoked <100 cigarettes in their lifetime; and smokers and former smokers at risk for lung cancer who are scheduled for a bronchoscopy were included in the study. Pregnant females; individuals with contraindications to fiberoptic bronchoscopy including hemodynamic instability; severe obstructive airway disease (as determined by spirometry); unstable angina, congestive heart failure; respiratory failure/hypoxemia; inability to protect airway; prior radiotherapy or chemotherapy to lungs or mediastinum; altered level of consciousness; or who inability to understand the consent form either due to mental status or language barriers were excluded from the study. The sample identity was blinded to the laboratory investigators and we accounted for age, gender and smoking status in the study design.

Blood Collection.

For exposure to CSE, a vein on the inside of the patient's elbow or the back of the patient's wrist was used for blood sampling. A tourniquet (tight band) was placed around the upper arm of the individual and the skin over the vein is usually cleaned with an antiseptic wipe. A needle is then inserted into the vein through the cleaned skin. The needle is connected either to a syringe, or directly to vacuumed sealed purple capped K2/K3 EDTA-coated tubes. (Sarstedt Aktiengesellschaft & Co., Numbrecht). After the required amount of blood, approximately 2-3 milliliters, is taken from the vein the needle is removed. The small wound is pressed on with cotton wool for a few minutes to stop the bleeding and prevent bruising.

Whole Blood CSE Exposure.

Frozen stocks of cigarette smoke extract were supplied by the lab of Andrew Dannenberg at Cornell University as described previously [11]. Concentrated 40.3 puffs/mL cigarette smoke extract was diluted to a working solution of 5 puffs/mL with PBS. 5 puffs/mL cigarette smoke extract (CSE) was administered directly into whole peripheral blood to a final concentration of 1 puff/mL CSE and allowed to incubate in a shaking 37° C. incubator for 3, 6, or 24 hours.

Immunofluorescence.

To determine the amount DNA damage after CSE incubation whole peripheral blood was administered to erythrocyte lysis buffer, cells were laid over poly-D-lysine-coated coverslips and fixed with 4% paraformaldehyde (Electron Microscopy Sciences) at room temperature as described previously [12]. Subsequently, cells were permeabilized with 0.5% Triton X-100 (Sigma), followed by 5 rinses in PBS. Blocking was done in aluminum-covered plates overnight at 4° C. in 10% FBS. Coverslips were then incubated for 1 hour at room temperature with mouse anti-phospho-Histone H2A.X (JW301 Upstate) Temecula, Calif. at a dilution of 1:400, Mouse anti-8-oxoguanine clone 483.15 (Upstate, MAB3560) at 1:250 or Rabbit anti-nitrotyrosine (Upstate, 06-284) at 1:200 then rinsed with 0.1% Triton X-100. Following a second 10% FBS blocking, cells were stained with FITC-conjugated anti-mouse IgG (Jackson ImmunoResearch, West Grove, Pa.) at a dilution of 1:150 and (1:200) for 1 hour at room temperature for samples with γH2AX primary and with 8-oxoguanine primary antibody respectively. Alexa 594-conjugated anti-rabbit IgG (Jackson ImmunoResearch) (1:200) was used for samples that were incubated with nitrotyrosine primary antibody. Coverslips were mounted onto slides using VECTASHIELD with DAPI (Vector Laboratories, Burlingame, Calif.). Foci were analyzed on a Zeiss automated microscope. At least 120 cells were counted per sample and cells with more than four distinct foci in the nucleus were considered positive for γH2AX.[13] Cells that exhibited elevated fluroscent intensity compared to background were considered positive for 8-oxoguanine, and Nitrotyrosine respectively. Positive cells were determined on a Zeiss automated microscope. Apoptotic cells, which have an approximate 10-fold increased in nuclear foci in damaged cells, were not included in analyses [13, 14]. Statistical analysis was done using a linear mixed model with repeated measures nested within an individual using STATA statistical analysis software.

Statistical Analyses.

Statistical analyses were done using bi-variate and multi-variate linear mixed models with repeated measures nested within an individual. According to (Afifi, A. A, Virginia Clark, and Susanne May. Computer-aided Multivariate Analysis. 4th ed. Boca Raton, Fla.: Chapman & Hall/CRC, 2004). Bi-variant interactions are considered interactions between two distinct variables to determine if any combination of factor levels can have a different linear effect on the dependent variable. (Afifi, A. A, Virginia Clark, and Susanne May. Computer-aided Multivariate Analysis. 4th ed. Boca Raton, Fla.: Chapman & Hall/CRC, 2004) Suggest these interaction models were assessed to determine if the linear relationship between a covariate and the dependent variable changes for different levels of a factor. Additionally, multi-variate linear mixed models were conducted to estimate a model with more than one outcome variable. Linear Mixed Models were utilized due to the ability to assess correlated and non-constant variability. Furthermore, these models provide the flexibility to assess not only the mean of a response variable, but its covariance structure as well. To further strengthen our data within these models categorical predictors of age and pack years smoke were assessed. The dependent variables were my genotoxic readouts of γH2AX, 8-oxoguanine, and nitrotyrosine. Measurements of each DNA damage parameter were conducted in each individual and assessed using STATA statistical analysis software.

### Results

Bi-variant modeling was used to assess if cancer versus non-cancer individuals, age, race, sex, past cancer history, pack years smoked, familial history of cancer, and history of harmful exposure are positive predictors of cigarette smoke extract induced γH2AX, 8-oxoguanine, and Nitrotyrosine formation in peripheral leukocytes over time. These assays were conducted in individuals with and without lung cancer that were exposed to cigarette smoke extract. In all assays percent positive cells were assessed in peripheral white blood cells via fluorescent microscopy.

### Markers of DNA Damage

H2AX is a member of the histone H2A protein family and becomes rapidly phosphorylated in the presence of a DNA damaging event. This rapid phosphorylation causes recruitment of DNA repair proteins to the site of the break and is detectable by specific antibodies to γH2AX. The formation of γH2AX a marker of double stranded breaks in peripheral leukocytes were counted in each individual.

8-oxoguanine is a mutagenic lesion caused by the interaction of a reactive oxygen species to DNA that causes G:C to T:A transversion mutations during replication.[15] Induction of 8-oxoguanine in peripheral leukocytes is an indication of increased ROS mediated DNA damage.

Nitrotyrosine is a biochemical marker for inflammation that is formed from nitric oxide-induced peroxynitrite interacting with other reactive nitrogen species to tyrosine residues of proteins [15, 16].

### Age

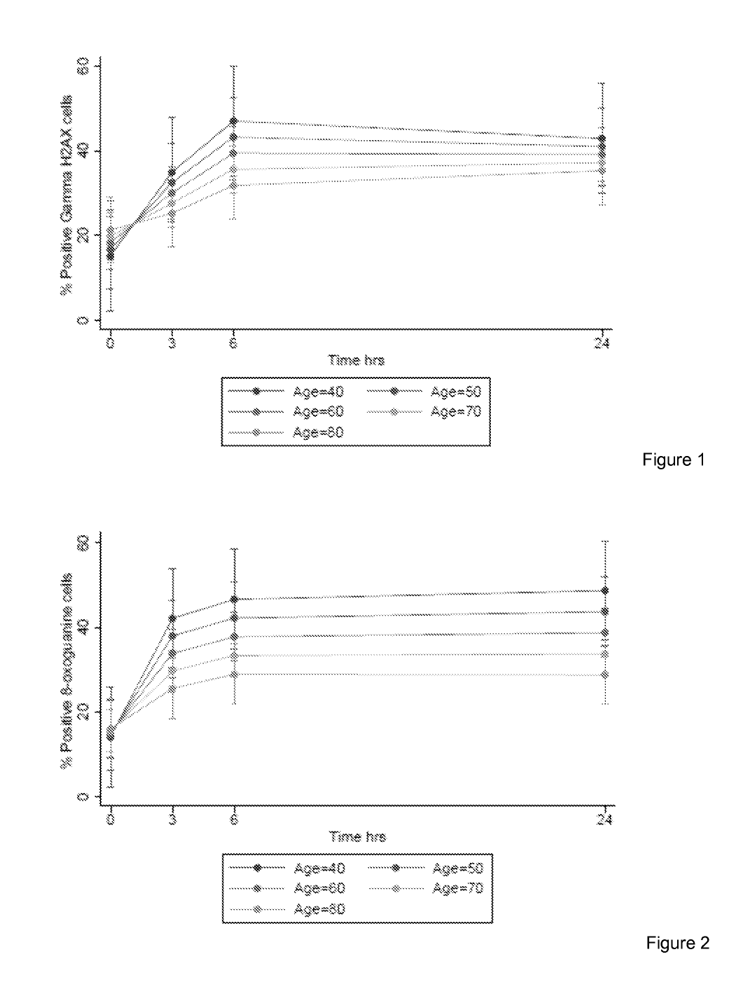

Age of the individual has been shown to be a contributing factor in cancer incidence.[17] We examined the role age plays in the assessment of the increased genotoxic susceptibility. At baseline formation of γH2AX are highest in individuals who are in their 80's (lowermost line) followed closely by individuals in their 70's (second line from bottom), 60's (middle line), 50's (second line from top) and the lowest for individuals who are in their 40's (uppermost line) (FIG. 1). Upon administration of cigarette smoke extract to the peripheral blood all groupings flipped causing a disordinal interaction. This interaction is described by observing individuals with highest baseline γH2AX foci formation, have the lowest amount of γH2AX foci formed at all other time points, and the lowest baseline γH2AX formation individuals have the highest amount of γH2AX foci formed at all other time points (FIG. 1). The contribution of age was a significant predictor of γH2AX-induced genotoxicity at p<0.0247. This suggests that age is a strong predictor of γH2AX-induced genotoxicity (FIG. 1).

A measure of ROS induced genotoxicity via 8-oxoguanine staining showed that at baseline there was very little difference in positive 8-oxoguanine staining in all age groups as indicated by the almost single data point. After 3 hrs of cigarette smoke extract incubation individuals who were in the 40's age range (uppermost line) had the highest amount of 8-oxoguanine staining (FIG. 2). This trend persisted throughout the 24 hr time course as did the lowered amount of 8-oxoguanine staining observed in the 50's (second highest line), 60's (central line), 70's (second lowest line), 80's (lowest line) year old groupings, respectively (FIG. 2). The data show that contribution of age being an indicator of 8-oxoguanine induced DNA damage was trending towards significant at p=0.0759 (FIG. 2).

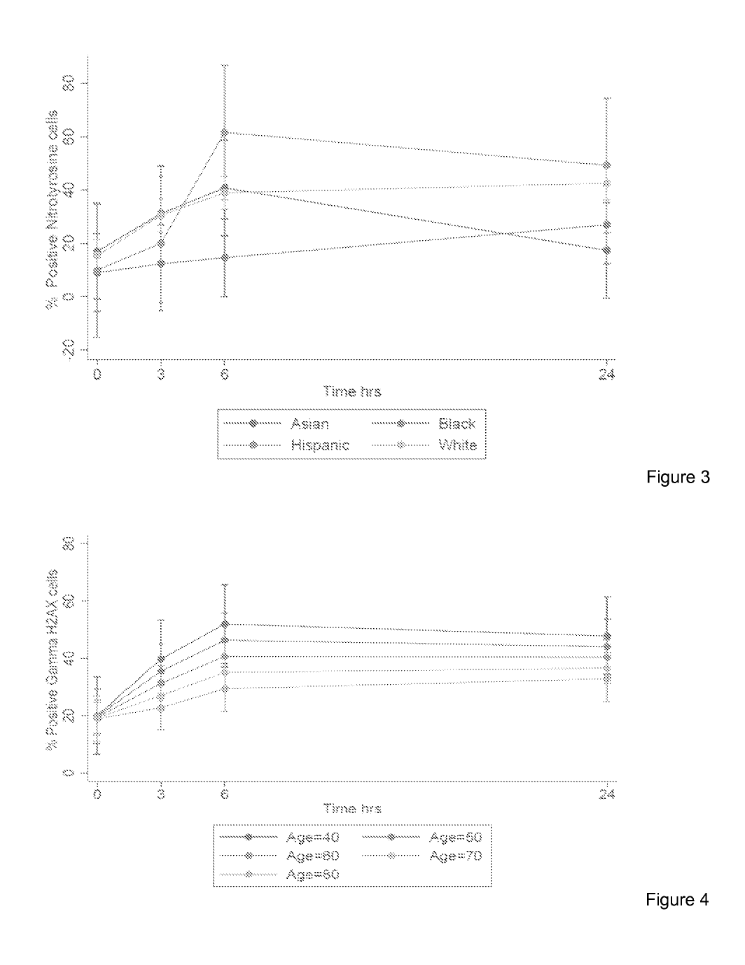

Racial demographic has been shown to be key predictor to aggressiveness and prevalence of lung cancer incidence in individuals.[18, 19] We examined the role race plays in the assessment of the increased genotoxic susceptibility. Using nitrotyrosine as an indicator of inflammation, we observed that at baseline Asian and White subjects have slightly higher amounts of positively stained nitrotyrosine cells compared to other ethnic groups. At 3 hrs of cigarette smoke extract incubation, we observe an increase in positively stained nitrotyrosine cells in all ethnic groups. This increase persists after 6 hr cigarette smoke extract induction but is observed highest in Hispanic individuals (FIG. 3). At 24 hrs of cigarette smoke extract incubation there is a continuous induction of positive nitrotyrosine stained cells compared to 6 hrs in White and Black individuals. Conversely in Hispanic and Asian individuals there is a decrease at 24 hr time point compared to the 6 hrs time point (FIG. 3). These observations were significant at p<0.0243 (FIG. 3).

Multi-variate modeling assessing models of interaction between time of extract incubation and age while controlling for sex, race, and pack years smoked. After assessing our bi-variant interactions we now wanted to establish our larger interaction models while controlling for variables that could have a collinear influence on our interaction. We assess γH2AX foci formation in the newest interaction model. At baseline we observe that there is little variation between the age groups. After administering cigarette smoke extract we detect an induction of positively stained cells in all groups at three hours.

The group that exhibited the highest accumulation of double strand breaks were the individuals who are in their 40's, followed by individuals who are in their 50's, individuals who are in their 60's, individuals in their 70's, and finally individuals who are in their 80's exhibited the lowest amount of γH2AX foci formation in peripheral leukocytes (FIG. 4). This trend persisted throughout the duration of the study at subsequent time points. After controlling for the variables of sex, race, smoking history measured by pack years in the patients a statistically significant positive interaction at p<0.0240 occurs between incubation time and age (FIG. 4). This suggests that an individual's age will positively affect the amount of accrued genotoxicity. This model also yielded a near statistically significant results in our controlled variable, race at p=0.0795 (FIG. 4).

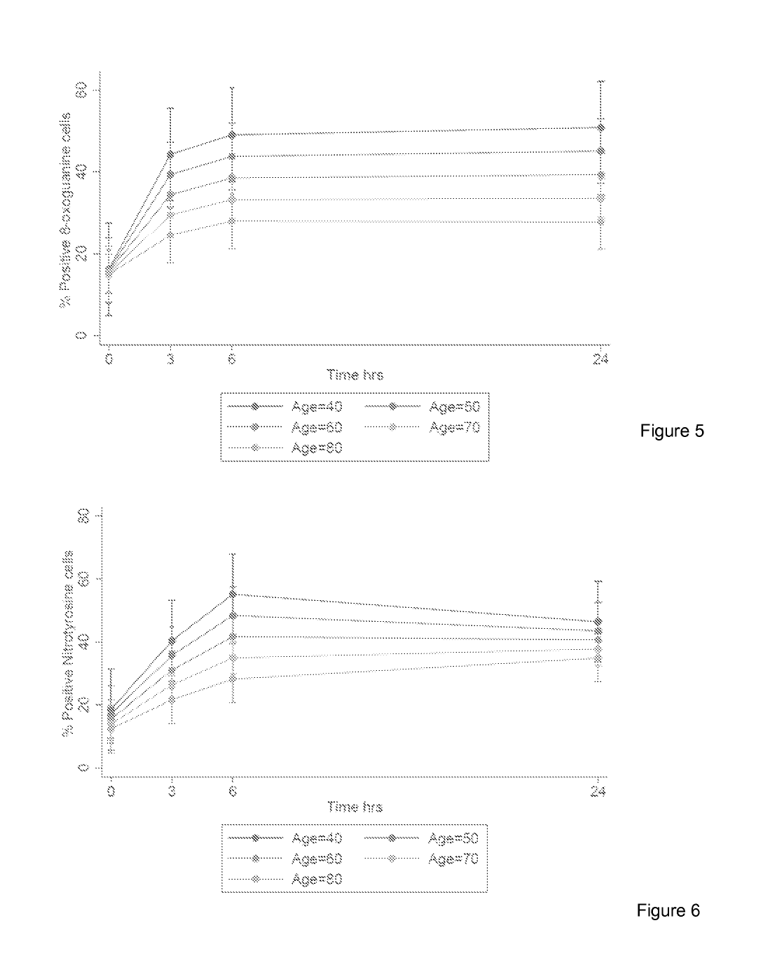

Assessing reactive oxygen species induced DNA damage in our multivariant model, we observed a very similar trend of DNA damage as our marker of double strand breaks. Very little distinguishing properties between the groups at baseline occurred. At 3 hrs, a clear induction in all groups occurred. The highest induction at this time point occurred in individuals who are in their 40's, followed by individuals who are in their 50's, individuals who are in their 60's, individuals in their 70's, and finally individuals who are in their 80's exhibited the lowest percent of positive cells (FIG. 5). This observation is trending towards significant at, p=0.0749. This multivariant model also yielded a statistically significant race variable at p<0.0012 (FIG. 5).

The marker of inflammation yielded results that were similar to the previously mentioned markers of DNA damage. At every time point throughout the incubation, the highest induction occurred in individuals who are in their 40's, followed by individuals who are in their 50's, individuals who are in their 60's, individuals in their 70's, and finally individuals who are in their 80's exhibited the lowest percent of positive cells (FIG. 6). This observation was not significant at, p=0.2179. Conversely, this multivariant model also yielded a statistically significant race variable at p<0.0012 (FIG. 6).

### History of Lung Cancer

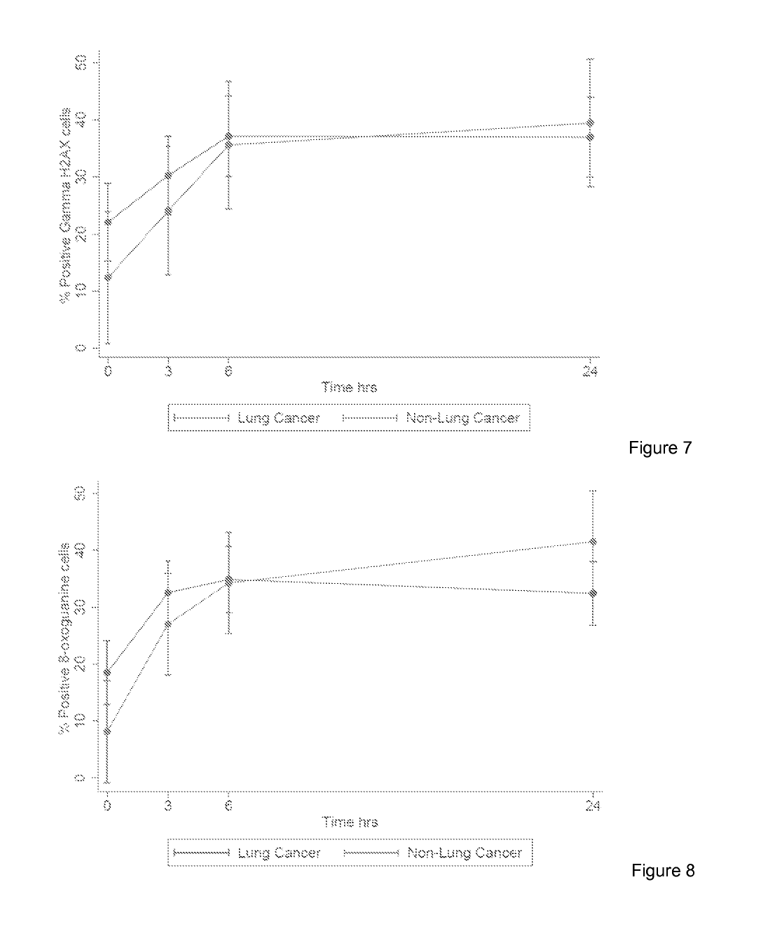

Multi-variate modeling assessing models of interaction between time of extract incubation and history of lung cancer while controlling for sex, race, and pack years smoked. Assessing cigarette smoke extract induced γH2AX between individuals who have lung cancer and individuals who do not have confirmed lung cancer. We observe at baseline individuals with lung cancer have a two-fold higher amount of positively stained γH2AX compared to individuals who do not have confirmed lung cancer (FIG. 7). This trend persisted and for 3 and 6 hrs of cigarette smoke extract incubation. On the other hand at 24 hrs individuals with no confirmed lung cancer exhibited an induction of positively stained γH2AX foci formed in peripheral leukocytes than individuals with lung cancer individuals with lung cancer (FIG. 7). This interaction was trending towards significant at p=0.0971 (FIG. 7).

Using 8-oxoguanine as a marker of DNA damage, we detect an induction in the baseline amounts of individuals with lung cancer have 2 fold higher amounts of positively stained γH2AX compared to individuals who do not have confirmed lung cancer (FIG. 8). This trend occurs at 3, and 6 hr cigarette smoke extract induced DNA damage. As observed with γH2AX an inverse of increased genotoxicity occurs and individuals who do not have confirmed lung cancer exhibit the highest induction of positively stained cells at 24 hrs cigarette compared individuals who do not have confirmed lung cancer (FIG. 8). This interaction was significant at p=0.0136 (FIG. 8).

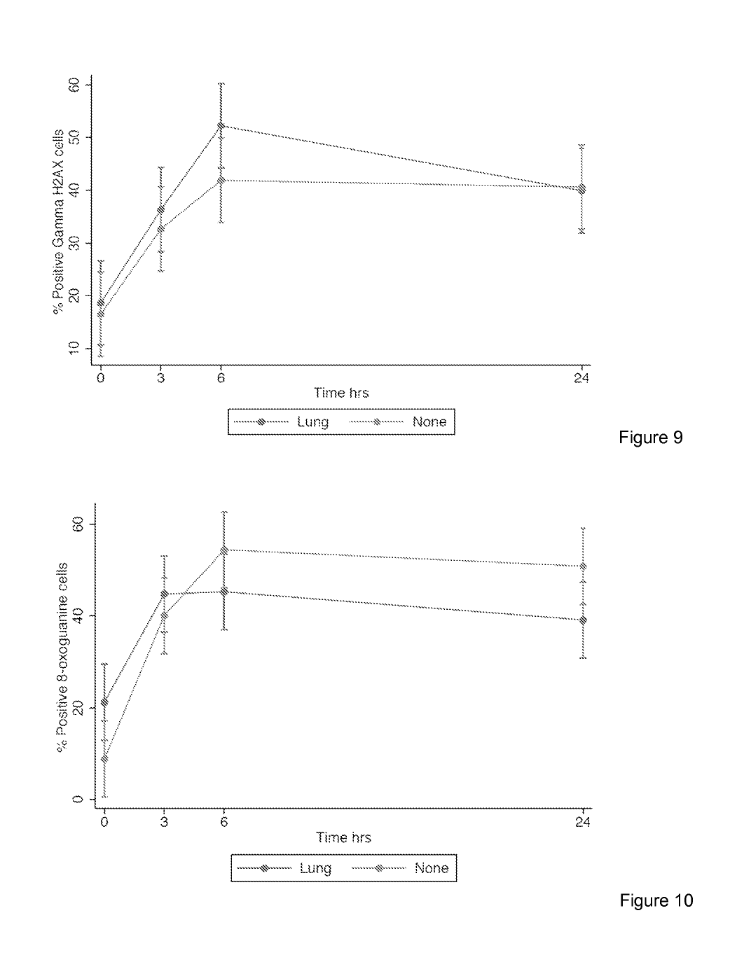

Multi-variate modeling assessing interaction between time of extract incubation and individuals who are 40-59 years of age and have a lung cancer and non-lung cancer controls while controlling for smoking history. Assessing cigarette smoke extract induced γH2AX between individuals who are 40-59 years of age and have lung cancer and individuals who do not have confirmed lung cancer. We observe at baseline individuals who are 40-59 years with lung cancer have a slightly higher amount of positively stained γH2AX compared to individuals who do not have confirmed lung cancer who are in the same age range (FIG. 9). This trend persisted and for 3 and 6 hrs of cigarette smoke extract incubation. At 6 hrs of cigarette smoke extract incubation this interaction is trending towards significant at 0.0826. At 24 hrs individuals who are 40-59 years with no confirmed lung cancer exhibited a near identical induction of positively stained γH2AX foci formed in peripheral leukocytes than individuals who are 40-59 years with lung cancer (FIG. 9). Overall this interaction was 0.3282 (FIG. 9).

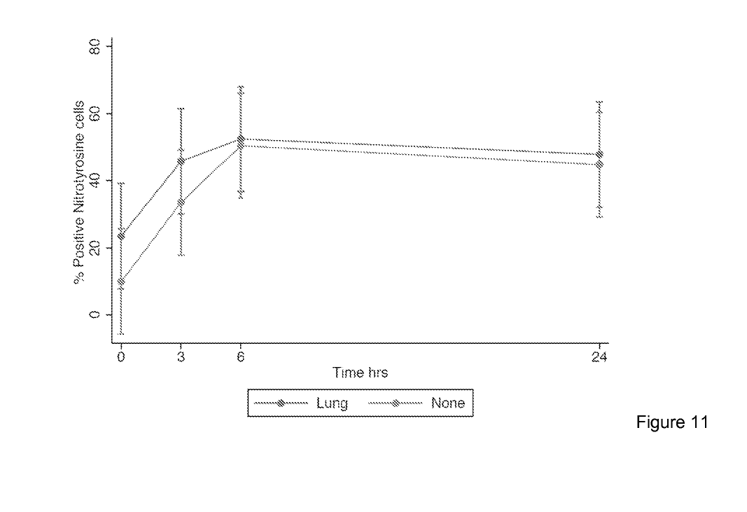

Multi-variate modeling using 8-oxoguanine as a marker of DNA damage we assessed the amount of ROS induced DNA damage in individuals who are 40-59 years of age and have a history of lung cancer or in non-lung cancer controls while controlling for smoking history. We detect about a two-fold induction of 8-oxoguanine positively stained cells at baseline in individuals with lung cancer compared to individuals who do not have confirmed lung cancer (FIG. 10). This trend persists at 3 hrs, but at 6 hr and 24 hr of cigarette smoke extract incubation there is an inverse of this trend and individuals who do not have confirmed lung cancer have higher 8-oxoguanine positively stained cells compared to individuals who do have lung cancer. This interaction was significant at p=0.0099 (FIG. 10).

Multi-variate modeling using nitrotyrosine as a marker of DNA damage we assessed the amount of positively stained cells in individuals who are 40-59 years of age and have a history of lung cancer or in non-lung cancer individuals while controlling for smoking history. We detect about a two-fold induction of nitrotyrosine positively stained cells at baseline in individuals with lung cancer compared to individuals who do not have confirmed lung cancer (FIG. 10). This trend persists throughout cigarette smoke extract incubation at 3, 6, and 24 hrs. This interaction is p=0.8293 (FIG. 11).

### DISCUSSION

Lung cancer is the leading cause of death from cancer in both men and women in the United States. [1, 2]. Although cigarette smoking is the predominating cause of lung cancer incidence only a subset of smoking individuals develop the disease. This suggests that genetic modulation of prominent factors may be leading to susceptibility in these individuals. [20] In this Example, we sought to establish biomarkers of susceptibility by assessing cigarette smoke extract induced DNA damage in patients with or without lung cancer. Peripheral blood leukocytes of these individuals were utilized and markers of DNA double strand breaks, reactive oxygen species induced DNA damage, and damage to nitrotyrosine residues caused by inflammation were assessed. As further contributions of susceptibility we assessed the contributions of age, race, gender, past cancer history, smoking history measured as pack years smoked, family cancer history, and previous chemical hazardous exposure history of these individuals that may lead to DNA damage. Cigarette smoke contains over 6000 chemicals many of which are known carcinogenic agents.[10] The carcinogenic compounds present in cigarette smoke are heterogeneous in nature and cause various intrinsic changes to the composition of many tissues in which they interact. Upon metabolism of many of the carcinogenic constituents in cigarette smoke water-soluble extracts of cigarette smoke are formed in some body compartments, such as blood, saliva, or fluid lining alveolar spaces, these extracts can contain active carcinogenic metabolites and can act on both cellular and extracellular compartments [21]. Thus addition of cigarette smoke extract into peripheral blood recapitulates a natural smoking environment and serves as a great tool to assess genotoxicity.

We utilized bi-variate and multi-variate models to assess if the age of an individual concomitantly with cigarette smoke extract incubation increases genotoxic susceptibility. Age of the individual has been shown to be a contributing factor in cancer incidence.[17] In addition according to 2009 statistics from the center for disease control the risk of developing lung cancer increases in age and is higher in men than it is in women. Furthermore, the center for disease control depicts that at the age of 60 there is an expected 2.27% and 1.72% increase of men and women to develop lung cancer sometime over a 10 year span, respectively. In our study we found that a positive interaction occurred between age and time of cigarette smoke extract incubation in both our bi-variant and multi-variant statistical models. Unexpectedly we observed a significant increase in the amount of positive γH2AX foci in younger individuals compared to older individuals. We also identified a near significant increase in 8-oxoguanine staining and a non-significant trend in nitrotryosine staining depicting similar increases in DNA damage in younger individuals. Thus, it is likely that the younger the patients are in which lung cancer develops the more sensitive they are to the genotoxicity of cigarette smoke. It is very interesting that the one of the individuals who did fall within this group was non-smoking and even more sensitive to the extract than any other patient. This highly sensitive individual was either exposed to passive tobacco smoke or the exposure to ambient air particulate matter air pollution, which has a similar consistency to cigarette smoke may have been enough to cause the lung cancer in this sensitive individual.

We also assessed the effect that smoking history has on DNA damage. Smoking tobacco is the major etiological risk factor for lung cancer development in current or former smokers.[22] Although smoking is the most prevalent cause of lung cancer 15% of lung cancer patients have never smoked and lung cancer in these non-smoking individuals comprise the seventh leading cause of mortality amongst solid tumors.[23] We sought to determine the interaction that smoking history has on our biomarkers of DNA damage. Using all three markers we see that non-smokers had high to moderate amounts of DNA damage that persisted throughout cigarette smoke extract incubation. We also see a trend that individuals with a longer smoking history had a tendency to clear the cigarette smoke extract induced DNA damage at faster rates than that of never smoking individuals. A possible rationale to this trend is that current and former smokers may have increased DNA repair enzymes, thus the recognition and removal of the damage is faster. Conversely, never smoking individuals do not have an up-regulation of these enzymes making the clearing of the damage occur at a slower rate than smoking or former smoking individuals. Furthermore, it is well established that genetic modulation of important detoxifying enzymes renders an individual to become more susceptible to lung cancer.[6, 24-26]. Although not assessed in our work it is a possibility that individuals who do not properly remove cigarette smoke induced DNA damage may, due to faulty repair systems, be more susceptible. It is established that individuals of certain racial demographics have been shown to exhibit increased incidence of lung cancer. [18, 19]. Thus, one can differentiate lung cancer patients, especially those in their 40s and 50s from non-cancer individuals by their genotoxicity profile to cigarette smoke extract.

Although we only observed a significant increase in one of the biomarkers in our bi-variant interaction model, when race is controlled for in our larger multi-variant model, we see the significant interaction race has on 8-oxoguanine, and nitrotyrosine induced DNA damage and a near significant induction of γH2AX. These data show that race has a significant interaction on susceptibility to cigarette smoke induced DNA damage. When assessing the specific races, it is shown that individuals of Hispanic ethnicity have the highest induction of DNA damage in all three biomarkers followed by individuals who have White ethnicity, Asian ethnicity, and lastly Black ethnicity. It is established that cigarette smoke and its many components can induce reactive oxygen species that cause mutagenic lesions that are normally repaired by specific DNA repair proteins.[26-28]. Recently it has been shown that SNPs in base excision repair genes in Hispanic and Black individuals increase the risk of developing lung cancer.[29]. Although the prevalence of lung cancer is second highest in Whites one study reveals that these individuals averaged the most lung cancer related surgical operations leading to a lower mortality rate than other ethnic groups with lower socioeconomic status. [9] A possible explanation of the relatively low increase in DNA damage in this group may be attributed to the higher amount of surgical procedures conducted. An increase of operation may be leading to better disease prognosis due to the fact that resection of tumors may cause a change in microenvironment thus slowing down the rate of new tumor formation and causing a decrease in DNA damage. It is established that individuals of Asian descent have relatively low smoking prevalence and lung cancer incidence. [30] Our data are consistent with this, and in our study we observed modest increases in cigarette smoke extract induced DNA damage in these individuals. Another plausible explanation to this modest increase may lie in the normal dietary intake of these individuals. [31] The protective impact of lifelong or early exposure to soy-derived isoflavones were associated with a 27% of risk reduction in lung cancer individuals. This increase of protection by a high-soy diet could possibly substantiate the modest cigarette smoke extract induced genotoxicity seen in this racial group.

Surprisingly in our study Black individuals consistently had lower amounts of cigarette smoke extract induced DNA damage. Yet, it is well established that Blacks have higher lung cancer incidence than any other racial group.[19]. Black men have the highest incidence of lung cancer as well as the highest mortality.[32] In a recent study, despite Black smokers having higher plasma cotinine per individual cigarette smoke exposure to nicotine and carcinogens per individual cigarette as assessed by urine biomarkers was similar or lower in Blacks compared to Whites.[18] This may suggest that Black individuals, although they may smoke more, may have lower exposure to cigarette carcinogens due to more frequent cigarettes but less intense smoking habits or higher clearance rates of the carcinogens. Our data are consistent with this study given the low amounts of cigarette smoke extract induced genotoxicity in this racial group. Furthermore, polymorphisms in DNA damage and repair genes may offer a plausible explanation for the observed modulation in response to genotoxicity in Blacks as well as other racial groups. In conjunction with race it has been well documented that there are sex disparities in cancer that make gender a significant variable in increased cancer incidence.[33, 34].

We observed that people with lung cancer displayed higher amounts of early susceptibility to DNA damage in all our biomarkers although this early damage waned as time progressed. These data depict a temporal susceptibility that may be present in lung cancer individuals and may suggest a target of therapeutic intervention. We also saw individuals with other cancers or a previous cancer history exhibited higher susceptibility to DNA damage in two of our three biomarkers. This data lead us to further interrogate of the individuals with a reported history of other cancer what cancer types would lead to increased susceptibility to cigarette smoke extract induced DNA damage.

The results of show significant interactions of age and race on cigarette smoke extract induced DNA damage as well as cancer versus non-cancer individuals, and positive trends in sex, previous personal cancer history, family history of cancer, and smoking history that is associated with lung cancer. Furthermore, in establishing our biomarkers we were able to detect increased susceptibility to cigarette smoke induced DNA damage in individuals with varying disease history and smoking status. We found that cancer patients have a two-fold higher level of DNA damage spontaneously compared to the control, however, they were able to more efficiently repair the damage which might be due to the fact that cigarette smoke exposure induces DNA repair. However, once we focused only on the 40 and 50-year old patients, they needed longer to repair the damage compared to the control. The nonsmoking lung cancer patient was even the most sensitive individual in our assay. This provides evidence for using these genotoxic assays as biomarkers in determining susceptibility to lung cancer, and opens the door to these biomarkers being used as possibly pre-screening tools to lung cancer predisposition and susceptibility in smoking and non-smoking individuals.

### REFERENCES

1. Thun, M. J., et al., J Natl Cancer lnst, 2006. 98(10): p. 691-9.
2. Halmos, B. and C. A. Powell, 2010. Am J Respir Crit Care Med. 184(3): p. 297-302.
3. Jemal, A., et al., CA Cancer J Clin, 2011. 61(2): p. 69-90.
4. Jemal, A., et al., Methods Mol Biol, 2009. 471: p. 3-29.
5. Littman, A. J., et al., Cancer Causes Control, 2004. 15(8): p. 819-27.
6. Lopez-Cima, M. F., et al., BMC Cancer, 2007. 7: p. 162.
7. Lv, J., et al., J Environ Monit, 2009. 11(7): p. 1368-74.
8. Mai, P., et al., Public Health Genomics, 2010. 13(7-8): p. 495-503.
9. LaPar, D. J., et al., Ann Thorac Surg, 2011. 92(2): p. 434-9.
10. Punturieri, A., et al., J Natl Cancer lnst, 2009. 101(8): p. 554-9.
11. Du, B., et al., Cancer Res, 2007. 67(18): p. 8966-72.
12. Goldstine, J. V., et al., DNA Repair (Amst), 2006. 5(4): p. 432-43.
13. Westbrook, A. M., et al., Cancer Res, 2009. 69(11): p. 4827-34.
14. Muslimovic, A., et al., Nat Protoc, 2008. 3(7): p. 1187-93.
15. Westbrook, A. M. and R. H. Schiestl, Cancer Res. 70(5): p. 1875-84.
16. Murata, M. and S. Kawanishi, Biochemical and Biophysical Research Communications, 2004. 316(1): p. 123-128.
17. Anderson, W. F., et al., JAMA, 2010. 303(17): p. 1723-8.
18. Benowitz, N. L., et al., Nicotine Tob Res. 13(9): p. 772-83.
19. Gallagher, C. M., et al., Spat Spatiotemporal Epidemiol, 2009. 1(1): p. 41-7.
20. Paz-Elizur, T., et al., J Natl Cancer lnst, 2003. 95(17): p. 1312-9.
21. Chen, H. W., et al., Chem Biol Interact, 2004. 150(3): p. 233-41.
22. Wen, J., et al., Chin J Cancer. 30(8): p. 551-8.
23. Torok, S., et al., Future Oncol. 7(10): p. 1195-211.
24. Fryer, A. A. and P. W. Jones, IARC Sci Publ, 1999(148): p. 303-22.
25. Goode, E. L., C. M. Ulrich, and J. D. Potter, Cancer Epidemiol Biomarkers Prev, 2002. 11(12): p. 1513-30.
26. Le Marchand, L., et al., Cancer Epidemiol Biomarkers Prev, 2002. 11(4): p. 409-12.
27. Lan, Q., et al., Carcinogenesis, 2004. 25(11): p. 2177-81.
28. Hecht, S. S., J Natl Cancer lnst, 1999. 91(14): p. 1194-210.
29. Chang, J. S., et al., Carcinogenesis, 2009. 30(1): p. 78-87.
30. Underwood, J. M., et al., Cancer, 2012. 118(7): p. 1910-8.
31. Yang, W. S., et al., Am J Clin Nutr, 2011. 94(6): p. 1575-83.
32. Lathan, C. S., et al., Cancer, 2010. 116(8): p. 1981-6.
33. Cook, M. B., et al., Cancer Epidemiol Biomarkers Prev, 2009. 18(4): p. 1174-82.
34. Murphy, G., et al., Int J Cancer, 2011. 128(7): p. 1668-75.

Throughout this application various publications are referenced. The disclosures of these publications in their entireties are hereby incorporated by reference into this application in order to describe more fully the state of the art to which this invention pertains.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for detection of predisposition to lung cancer in a subject, the method comprising:
(a) incubating a test sample of peripheral leukocytes from the subject with cigarette smoke extract;
(b) assaying the test sample for a marker of DNA damage;
(c) detecting an increase in the marker of DNA damage in the test sample relative to a control sample; and
(d) determining the presence of predisposition to lung cancer when an increased amount of the marker is present in the test sample compared to the control sample.

2. The method of claim 1, wherein the marker of DNA damage is single- and/or double-stranded DNA breaks in leukocytes.

3. The method of claim 1, wherein the measuring comprises an immunoassay for γ-H2AX, nitrotyrosine, or 8-oxoguanine.

4. The method of claim 1, wherein the marker of DNA damage is oxidative DNA damage in leukocytes.

5. The method of claim 4, wherein the measuring comprises an enzyme hOgg1-modified comet assay or an immunoassay for 8-oxoguanine.

6. The method of claim 1, wherein the marker of DNA damage is nitrotyrosine oxidation activity.

7. The method of claim 6, wherein the measuring comprises an immunoassay for protein nitrotyrosine in leukocytes.

8. The method of claim 1, wherein the peripheral leukocyte is a lymphocyte or a monocyte.

9. The method of claim 1, wherein the sample of peripheral leukocytes is obtained from peripheral blood, or fluid of a body cavity.

10. The method of claim 9, wherein the fluid of a body cavity is pleural, peritoneal, cerebrospinal, mediastinal, or synovial fluid.

11. A method for detection of lung cancer in a subject, the method comprising:
(a) incubating a test sample of peripheral leukocytes from the subject with cigarette smoke extract;
(b) assaying the test sample for a marker of DNA damage;
(c) detecting an increase in the marker of DNA damage in the test sample relative to a control sample; and
(d) determining the presence of lung cancer when an increased amount of the marker is present in the test sample compared to the control sample.

12. The method of claim 11, wherein the marker of DNA damage is single- and/or double-stranded breaks in leukocytes.

13. The method of claim 12, wherein the measuring comprises an immunoassay for γ-H2AX and/or an alkaline comet assay.

14. The method of claim 11, wherein the marker of DNA damage is oxidative DNA damage in leukocytes.

15. The method of claim 14, wherein the measuring comprises an enzyme hOgg1-modified comet assay or an immunoassay for 8-oxoguanine.

16. The method of claim 11, wherein the marker of DNA damage is nitric oxide-mediated oxidation activity.

17. The method of claim 16, wherein the measuring comprises an immunoassay for protein nitrotyrosine in leukocytes.

18. The method of claim 11, wherein the peripheral leukocyte is a lymphocyte or a monocyte.

19. The method of claim 11, wherein the sample of peripheral leukocytes is obtained from peripheral blood, or fluid of a body cavity.

20. The method of claim 19, wherein the fluid of a body cavity is pleural, peritoneal, cerebrospinal, mediastinal, or synovial fluid.
